(19) United States
(12) Patent Application Publication
(10) Pub. No.: US 2025/0326724 A1
(43) Pub. Date: Oct. 23, 2025

(54) PROCESS FOR THE PREPARATION OF 4-SUBSTITUTED 2-OXAZOLIDINONES

(71) Applicant: SYNGENTA CROP PROTECTION AG, Basel (CH)

(72) Inventors: Denis GRIBKOV, Münchwilen (CH); Andreas UNSINN, Muttenz (CH); Bjoern ANTELMANN, Münchwilen (CH); Antonia LUETTIN, Münchwilen (CH)

(73) Assignee: SYNGENTA CROP PROTECTION AG, Basel (CH)

(21) Appl. No.: 18/870,066

(22) PCT Filed: May 23, 2023
(86) PCT No.: PCT/EP2023/063798

§ 371 Date: Nov. 27, 2024

(30) Foreign Application Priority Data
May 30, 2022 (EP) 22176222.2

(51) Int. Cl. C07D 263/24; C07D 261/04

(52) U.S. Cl. CPC C07D 263/24; C07D 261/04

(57) ABSTRACT

The present invention relates to a process for the preparation of a compound of formula (II) wherein M is selected among Na, K and Li, by reacting a compound of formula (I) wherein R¹ is selected among hydrogen, Na, K and Li, with a base, a reagent, and optionally an organic solvent, characterized in that the base is a metal salt of alkoxide.

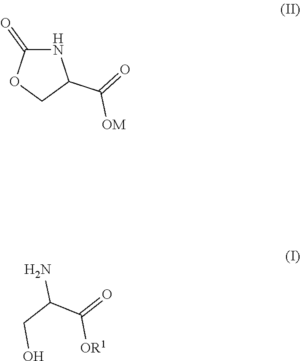

The present invention relates to novel method of producing 4-substituted 2-oxazolidinones, which are intermediates useful in the preparation of 2-substituted cycloserines.

2-substituted cycloserines are useful in the preparation of certain insecticidally active compounds, for example those described in WO2011/067272 and WO2012/163959. Furthermore, the preparation of 4-substituted 2-oxazolidinones described in WO2015/166094 does not provide an optimized yield and they are not easily isolating.

Therefore, there is still a need to improve the chemical yield of the preparation of 4-substituted 2-oxazolidinones, while guaranteeing an easier separation, especially for large scale production.

The aim of the present invention is to overcome the problems of the prior art techniques by proposing a process for the preparation of 4-substituted 2-oxazolidinones, which presents an optimized yield and/or an optimized purity, while guaranteeing an easier isolation that is fully scalable to manufacturing scale.

To this end, an object of the present invention is to provide a process for the preparation of a compound of formula II

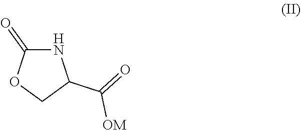

wherein M is selected among Na, K and Li,
by reacting a compound of formula I

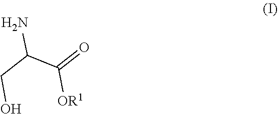

wherein R¹ is selected among hydrogen, Na, K and Li,
with a base, a reagent, and optionally an organic solvent, characterized in that the base is a metal salt of alkoxide.

The compound of formula II is a metal salt of 2-oxooxazolidine-4-carboxylic acid, and more preferably a potassium salt of 2-oxooazolidine-4-carboxylic acid.

In a preferred embodiment, the compound of formula II can have the following structure:

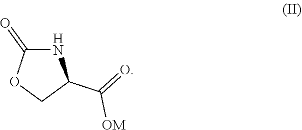

In a preferred embodiment, the compound of formula I can have the following structure:

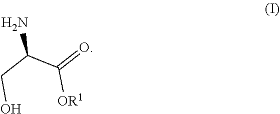

In the present invention, the metal salt of alkoxide is more particularly a strong base. The metal salt of alkoxide can be an alkali metal salt of C₁-C₅ alkoxide, which can be for example selected among potassium methoxide, sodium methoxide, lithium methoxide, sodium ethoxide, sodium tert-pentoxide, sodium tert-butoxide, potassium tert-butoxide, and any mixture thereof.

More preferably, the metal salt of alkoxide is a non-aqueous base. In a particular embodiment, the process for the preparation of a compound of formula II does not include any aqueous base, such as for example it does not include aqueous hydroxide base.

In the process according to the present invention, the amount of the base can be from 0.01 to 10 molar equivalents, preferably from 0.01 to 5 molar equivalents, preferably from 0.05 to 3.0 molar equivalents, and more preferably from 0.1 to 2 molar equivalents. The expression “molar equivalents” related to the base is based on the number of moles (mol) of the compound of formula I.

The reagent according to the present invention can comprise any suitable reagent well-known in the art. For example, the reagent can be selected among an organic carbonate, a halo-carbonate, and any mixture thereof.

The organic carbonate can be selected among an aryl-carbonate, an alkyl-carbonate, an aryl-alkyl-carbonate, and any mixture thereof. For example:
the aryl-carbonate can be diphenylcarbonate;
the alkyl-carbonate can be selected among dimethyl carbonate, diethyl carbonate, ethylene carbonate, propylene carbonate, and trimethylencarbonate;
the aryl-alkyl-carbonate can be methyl phenyl carbonate.

The halo-carbonate can be preferably a chloro-carbonate. For example, the halo-carbonate can be selected among phosgene or a derivative thereof. The phosgene derivatives can be for example diphosgene, triphosgene, methyl chloroformate, ethyl chloroformate, or benzylchloroformate.

The use of organic carbonate is preferred in the process according to the present invention, in order to limit the toxicity of the reagent, and more preferably dimethyl carbonate.

In the process according to the present invention, the amount of the reagent can be from 0.1 to 10 molar equivalents, preferably from 0.5 to 5 molar equivalents, preferably from 0.5 to 2.0 molar equivalents, and more preferably from 0.5 to 1.5 molar equivalents. The expression “molar equivalents” related to the reagent is based on the number of moles (mol) of the compound of formula I.

The organic solvent according to the present invention can comprise any suitable organic solvent well-known in the art, and more preferably an alcohol. For example, the organic solvent can be selected among methanol, ethanol, propanol, isopropanol, butanol, t-butanol, t-amyl alcohol, toluene, tetrahydrofuran, 2-methyl-tetrahydrofuran, and any mixture thereof. The reagent according to the present invention can be used as solvent, or can be used in a mixture with said organic solvent.

In the process according to the present invention, the amount of the organic solvent can be from 1 to 200 molar equivalents, preferably from 1 to 100 molar equivalents, and more preferably from 1 to 20 molar equivalents. The expression “molar equivalents” related to the organic solvent is based on the number of moles (mol) of the compound of formula I.

The process according to the present invention can further comprise a crystallisation step, and optionally then a separation step. More particularly, once the compound of formula II is obtained, said compound of formula II can be crystallized and then separated.

The separation step aims at removing base and reagent, and optionally solvent, used in excess. This separation step can be carried out by techniques well-known in the art such as for example by distillation, decantation, centrifugation or filtration (e.g. in using a centrifuge, a nutsche filter, a candle filter, or a pocket filter), or a combination of these techniques, and more preferably by filtration.

The crystallisation step can be carried out by techniques well-known in the art. The compound of formula II can crystallize during the reaction, or the crystallisation can be triggered by adding seed crystals of the compound of formula II during or after the reaction and/or by adding an anti-solvent. An anti-solvent is typically a solvent in which the compound of formula II is not soluble at all, such as for example methylisobutylketone or toluene. The crystallisation can also be initiated by concentrating the reaction mixture by distillation. The isolated compound of formula II can be dried by techniques well-known in the art. Typically the drying step can be done at elevated temperature and under vacuum, such as for example a temperature ranged from 30 to 100° C., and under a pressure ranged from 500 to 1 mbar, in dryers like paddle dryers, conical dryers, or filter dryers.

Another object according to the present invention relates to a process for the preparation of a compound of formula III

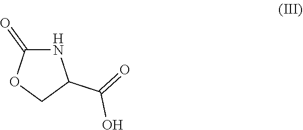

by reacting the compound of formula II with an acid in the presence of a solvent.

In a preferred embodiment, the compound of formula III can have the following structure:

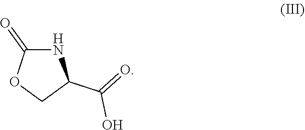

More particularly, this another object relates to the process for the preparation of the compound of formula II according to the present invention, wherein it can further comprise the step of reacting the compound of formula II with an acid in the presence of a solvent, to prepare a compound of formula III.

In a particular embodiment, after the crystallisation of the compound of formula II, the compound of formula III can be obtained without any separation step of the compound of formula II. More particularly, solvent can be exchanged by distillation, which is a technic well-known in the art.

In another particular embodiment, after the crystallisation of the compound of formula II, the compound of formula III can be obtained by filtering-off compound of formula II, washing the separated compound of formula II with suitable solvent and re-suspending compound of formula II in a suitable solvent before continuing the preparation of compound of formula III. Suitable solvents can be an organic solvent described thereafter.

In the preparation of the compound of formula III, the acid can be more particularly a strong acid, which can be selected among hydrochloric acid (HCl), sulfuric acid (H₂SO₄), hydrobromic acid (HBr), trifluoroacetic acid, methane sulfonic acid, perchloric acid and any mixture thereof. Preferably, the acid can be selected among hydrochloric acid, sulfuric acid, and any mixture thereof.

The acid can be anhydrous acid, such as HCl gas, 98% H₂SO₄; aqueous acid, such as hydrochloric acid and preferably concentrated hydrochloric acid with concentrations between 30 and 35%; or solutions in organic solvents, such as HCl in methanol, HCl in dioxane, HBr in acetic acid. If aqueous acid is used, water can be removed by azeotropic distillation.

The amount of the acid can be from 0.05 to 5 molar equivalents, preferably from 0.1 to 2.0 molar equivalents, and more preferably from 0.5 to 1.5 molar equivalents. The expression “molar equivalents” related to the acid is based on the number of moles (mol) of the compound of formula II.

In the preparation of the compound of formula III, the solvent can comprise any suitable solvent well-known in the art, and especially any solvent wherein the compound of formula III is soluble and the salt of the acid (used to prepare the compound of formula III) is not soluble.

For example, the solvent can be an organic solvent, more preferably selected among methyl isobutyl ketone, methyl ethyl ketone, acetone, 2-pentanone, propionic acid, acetic acid, tetrahydrofuran, 2-methyltetrahydrofuran, dioxane, methyl acetate, ethyl acetate, butyl acetate, dimethyl carbonate, ethylene carbonate and any mixture thereof.

In a preferred embodiment, the solvent used to obtain the compound of formula III can be methyl isobutyl ketone, acetone, a mixture of methyl isobutyl ketone and acetone, 2-pentanone, a mixture of 2-pentanone and acetone, methyl ethyl ketone, a mixture of 2-pentanone and methyl ethyl ketone, propionic acid, or acetic acid.

A small amount of water (typically 2-5% by weight) can be added to the solvent to increase solubility of the compound of formula III. Also, for better solubility of the compound of formula III, elevated temperatures are preferred, such as ranged from 50 to 100° C.

In another embodiment, solvents in which the compound of formula III is only partially soluble or insoluble at elevated temperatures (at least 50° C.) can be used such as xylene or chlorobenzene. In this case, the compound of formula III can be dissolved at a later stage, such as during filtration, in using an appropriate solvent wherein the compound of formula III is soluble and the salt of the acid (used to prepare the compound of formula III) is not soluble, such as an organic solvent as described above.

In the preparation of the compound of formula III, the amount of the solvent can be from 1 to 200 molar equivalents, and preferably from 5 to 100 molar equivalents. The expression “molar equivalents” related to the solvent is based on the number of moles (mol) of the compound of formula II.

The process for the preparation of a compound of formula III can further comprise a separation step and then optionally a crystallisation step, and optionally another separation step. More particularly, once the compound of formula III is obtained, said compound of formula III can be separated and then crystallized. If aqueous acid is used, water can be removed by azeotropic distillation, preferably before and/or during the separation step.

The separation step aims at removing the salt of the acid used to prepare the compound of formula III. This separation step can be carried out by techniques well-known in the art such as for example by decantation, centrifugation or filtration (e.g. in using a centrifuge, a nutsche filter, a candle filter, or a pocket filter).

The crystallisation step can be carried out by techniques well-known in the art. For example, the compound of formula III can be crystallised by cooling the solution typically at a temperature ranged from 100 to ˜10° C., and preferably from 80 to 0° C.; and/or by evaporating the solvent typically at a temperature ranged from 30 to 80° C., with or without vacuum.

The obtained solid of compound of formula III can be separated from solvent used during the crystallisation. This separation step can be carried out by techniques well-known in the art such as for example by distillation, decantation, centrifugation or filtration (e.g. in using a centrifuge, a nutsche filter, a candle filter, or a pocket filter), or a combination of these techniques.

The isolated compound of formula III can be dried by techniques well-known in the art. Typically the drying step can be done at elevated temperature and under vacuum, such as for example a temperature ranged from 30 to 100° C., and under a pressure ranged from 500 to 1 mbar, in dryers like paddle dryers, conical dryers, or filter dryers.

Another object of the present invention relates to a compound of formula Ia

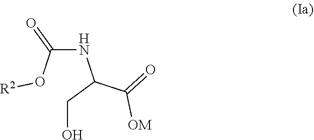

wherein R² is selected among C₁₋₄alkyl, phenyl, benzyl, C₂H₄OH, C₃H₆OH, CHCH₃CH₂OH, and CH₂CHCH₃OH; and M is selected among Na, K and Li. R² can be preferably C₁₋₄alkyl, and more preferably methyl.

In a preferred embodiment, the compound of formula Ia can have the following structure:

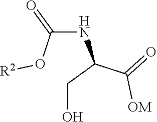

The compound of formula Ia can be formed as an intermediate, during the process for the preparation of a compound of formula II.

Another object according to the present invention relates to a process for the preparation of a compound of formula VI

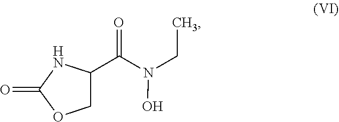 [SMILES: CCN(O)C(=O)C1COC(=O)N1]

including the process for the preparation of the compound of formula II according to the present invention and/or including the process for the preparation of the compound of formula III according to the present invention. More particularly, this another object relates to the process for the preparation of the compound of formula II according to the present invention and/or the process for the preparation of the compound of formula III according to the present invention, wherein it can further comprise the preparation of a compound of formula VI.

The compound of formula VI can be prepared for example according to WO2015/166094, which is incorporated herein by reference, shown in Scheme 2 on page 27.

More particularly, the compound of formula VI can be prepared by reacting a compound of formula III obtained by the process according to the present invention, with a compound of formula V

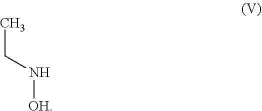

Preferably, the reaction includes preparing the corresponding acid halide (preferably acid chloride) of the compound of formula III, which is the compound of formula IV

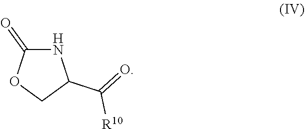

wherein R¹⁰ is halogen, to facilitate the conversion to the compound of formula VI.

The acid halide (i.e. the compound of formula IV), wherein R¹⁰ is halogen, can be prepared from the compound of formula III under conditions well known to the person skilled in the art, such as by treatment with thionyl chloride, oxalyl chloride, phosgene, diphosgene or triphosgene. Alternatively, the compound of formula IV, wherein R¹⁰ is halogen, can be prepared from an alkali metal (Li, Na, K) salt of compound of formula III, which is the compound of formula II, by treatment with oxalyl chloride, thionyl chloride, phosgene, diphosgene or triphosgene in the absence or in the presence of a catalyst and/or a phase transfer catalyst. Suitable catalyst includes, but are not limited to dimethyl formamide, dimethyl acetamide, N-methyl pyrrolidone. Suitable phase transfer catalysts include, but are not limited to tetrabutylamonium chloride, tetrabutylamonium bromide, triethylbenzylamonium chloride, Aliquat® 336 and (1-hexadecyl)trimethylamomnium bromide. More particularly, the compound of formula VI can be prepared by reacting a compound of formula II obtained by the process according to the present invention.

In a particular embodiment, after the crystallisation of the compound of formula III, the compound of formula IV can be obtained without any separation step of the compound of formula III.

In another particular embodiment, after the crystallisation of the compound of formula II, the compound of formula IV can be obtained by the process according to the present invention without further isolating the compound of formula II and/or the compound of formula III, in a dried solid form.

Another object of the present invention relates to a process for the preparation of a compound of formula VIII

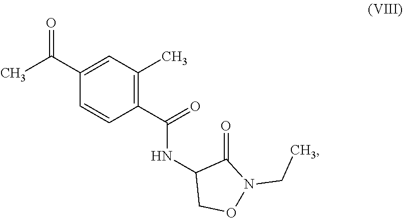 [SMILES: CCN1OCC(NC(=O)c2ccc(C(C)=O)cc2C)C1=O]

including the process for the preparation of the compound of formula II according to the present invention and/or in using the process for the preparation of the compound of formula III according to the present invention. More particularly, this another object relates to the process for the preparation of the compound of formula II according to the present invention and/or the process for the preparation of the compound of formula III according to the present invention, wherein it can further comprise the preparation of a compound of formula VIII, especially after the preparation of a compound of formula VI.

In a preferred embodiment, the compound of formula VIII can have the following structure:

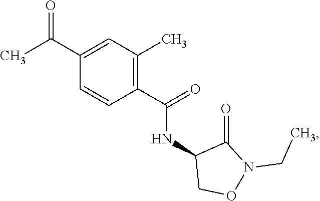 [SMILES: CCN1OC[C@@H](NC(=O)c2ccc(C(C)=O)cc2C)C1=O]

The compound of formula VIII can be prepared for example according to WO2015/166094, which is incorporated herein by reference.

More particularly, the compound of formula VIII can be prepared by converting the compound of formula VI to a compound of formula VII

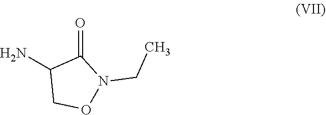

with a base, and more preferably with an aqueous solution of a base. For example, said base can be an aqueous solution of sodium hydrogen carbonate, sodium carbonate, and/or sodium hydroxide.

In a preferred embodiment, the compound of formula VII can have the following structure:

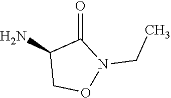

Then the process may include reacting the compound of formula VII with a second compound, wherein the second compound comprises a carboxylic acid, acid halide, ester or thioester functional group, and the reaction comprises reacting the amine functional group of the compound of formula VII with the carboxylic acid, acid halide, ester or thioester functional group of the second compound such that the compound of formula VII is coupled to the second compound via an amide functional group, or wherein the second compound comprises a dicarbonate group, and the reaction comprises reacting the amine functional group of the compound of formula VII with the dicarbonate group of the second compound, such that the compound of formula VII is coupled to the second compound via a carbamate functional group.

This process is well known in the art and for example is described in WO2015166094, which is incorporated herein by reference.

Another object of the present invention relates to the preparation of a compound of formula XI

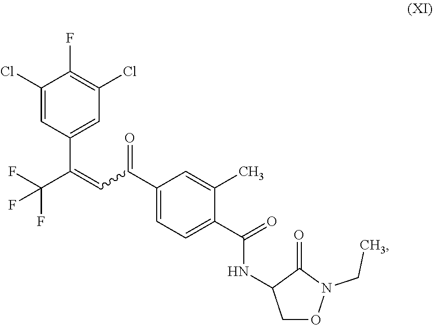 [SMILES: CCN1OCC(NC(=O)c2ccc(C(=O)C=C(c3cc(Cl)c(F)c(Cl)c3)C(F)(F)F)cc2C)C1=O]

including the process for the preparation of the compound of formula II according to the present invention and/or including the process for the preparation of the compound of formula III according to the present invention. More particularly, this another object relates to the process for the preparation of the compound of formula II according to the present invention and/or the process for the preparation of the compound of formula III according to the present invention, wherein it can further comprise the preparation of a compound of formula XI, especially after the preparation of a compound of formula VIII.

In a preferred embodiment, the compound of formula XI can have the following structure:

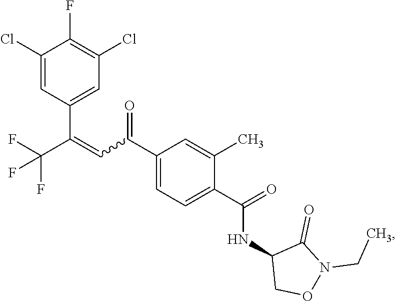 [SMILES: CCN1OC[C@@H](NC(=O)c2ccc(C(=O)C=C(c3cc(Cl)c(F)c(Cl)c3)C(F)(F)F)cc2C)C1=O]

The preparation of the compound of formula XI is based on a dehydration reaction, said reaction being well-known in the art. The compound of formula XI can be prepared, for example according to WO2011/067272, in particular shown in Scheme 3 on pages 18-19. More particularly, the compound of formula XI can be prepared by reacting a compound of formula X

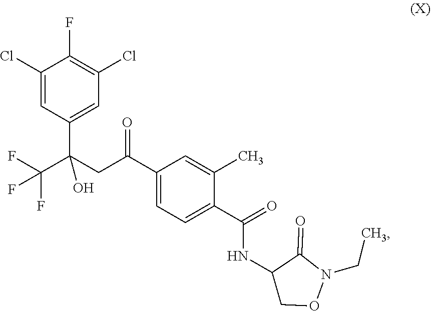 [SMILES: CCN1OCC(NC(=O)c2ccc(C(=O)CC(O)(c3cc(Cl)c(F)c(Cl)c3)C(F)(F)F)cc2C)C1=O]

in an organic solvent such as hexane, heptane, methycyclohexane, toluene, xylene, chlorobenzene, o-dichlorobenzene, dichloroemethane, dioxane, tetrahydrofuran, 2-methyltetrahydrofuran, cyclopentylethylether, anisole, acetonitrile, propionitrile, butyronitrile, benzonitrile, or any combinations thereof; with a base such as triethylamine, tri-n-butylamine, pyridine, or any combinations thereof; a dehydration agent such as phosgene, thionyl chloride, acetic anhydride, acetyl chloride, methanesulfonyl chloride, oxalyl chloride, methyl chloroformate, ethyl chloroformate, or any combinations thereof; and a catalyst such as aminopyridine catalyst which can be for example 4-dimethylaminopyridine or 4-pyrrolidinopyridine. Said mixture can be stirred in a reactor for about 10 minutes to 96 hours, and preferably about 1 to 20 hour(s), usually at 0 to 150° C., preferably at 0 to 20° C., and more preferably at 0 to 10° C.

In a preferred embodiment, the compound of formula X can have the following structure:

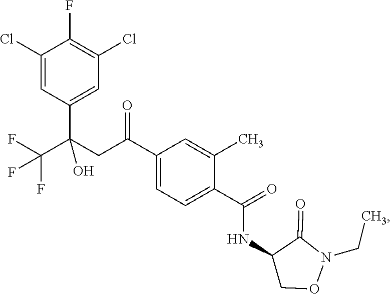 [SMILES: CCN1OC[C@@H](NC(=O)c2ccc(C(=O)CC(O)(c3cc(Cl)c(F)c(Cl)c3)C(F)(F)F)cc2C)C1=O]

The compound of formula XI can be isolated with work-up conditions well-known in the art, in separating the base, the dehydration agent, the catalyst or its respective reaction products from the compound of formula XI.

In a first embodiment, the compound of formula XI according to the present invention can comprise the E-configuration compound of formula XI, and optionally the Z-configuration compound of formula XI. More particularly, the compound of formula XI can comprise a E/Z ratio from 90:10 to 100:0, preferably from 95:5 to 100:0, and more preferably from 99:1 to 100:0.

In a second embodiment, the compound of formula XI according to the present invention can comprise a R/S ratio from 50:50 to 100:0, preferably from 90:10 to 100:0, and more preferably from 95:5 to 100:0.

In a third embodiment, the compound of formula XI according to the present invention can comprise the first embodiment and the second embodiment.

The preparation of the compound of formula X as described beforehand, is based on an aldol reaction, said reaction being well-known in the art. More particularly, the compound of formula X can be prepared by reacting an aromatic ketone compound of formula IX

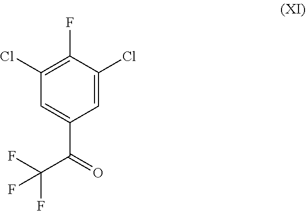 [SMILES: O=C(c1cc(Cl)c(F)c(Cl)c1)C(F)(F)F]

with the compound of formula VIII, in the presence of a base, with or without a solvent.

The base can be for example triethylamine, trimethylamine, diethylamine, tert butylamine, pyridine, 1,8-diaza (5,4,0)-7-bicycloundecene, potassium carbonate, or any combination thereof. The solvent can be for example selected among toluene, xylene, chlorobenzene, dichlorobenzene, anisole, dimethoxybenzene, dioxane, tetrahydrofuran, 2-methyltetrahydrofuran, dimethylcarbonate, ethyl acetate, methoxyethyl acetate, and any combinations thereof.

The equilibrium of the reaction can be shifted towards the compound of formula X by adjusting the amount of solvent in such a way that the reaction is run as concentrated as possible with sufficient mixing. The mixture can be a homogenous solution or can be a slurry. Said mixture can be stirred in a reactor for about 1 to 150 hours, and preferably about 1 to 96 hour(s), usually at 0 to 150° C., preferably at 20 to 60° C., and more preferably at 30 to 50° C.

The compound of formula X can be isolated or can be used without further work-up as such, to generate the compound of formula XI.

Another object according to the present invention relates to a process for the preparation of a compound of formula XII or an enriched composition comprising a compound of formula XII

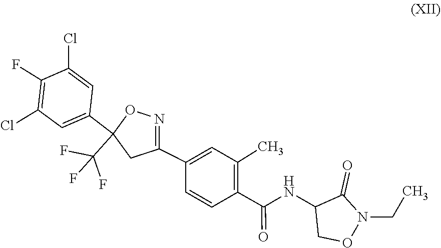 [SMILES: CCN1OCC(NC(=O)c2ccc(C3=NOC(c4cc(Cl)c(F)c(Cl)c4)(C(F)(F)F)C3)cc2C)C1=O]

including the process for the preparation of the compound of formula II according to the present invention and/or including the process for the preparation of the compound of formula III according to the present invention. More particularly, this another object relates to the process for the preparation of the compound of formula II according to the present invention and/or the process for the preparation of the compound of formula III according to the present invention, wherein it can further comprise the preparation of a compound of formula XII, especially after the preparation of a compound of formula XI.

In a preferred embodiment, the compound of formula XII can have the following structure:

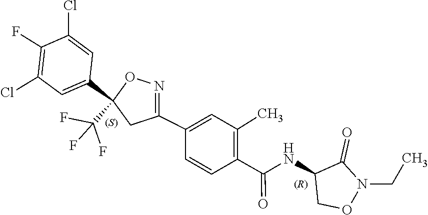

which is the isomer (5S,4R) of the compound of formula XII (4-[(5S)-5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide). The preparation of an enriched composition can comprise the compound of formula XII (5S,4R) and at least one of the isomers of the compound of formula XII selected among isomer (5S,4S), isomer (5R,4R), isomer (5R,4S), and any combinations thereof. The isomer (5S,4S) is 4-[(5S)-5-(3,5-dichloro-4-fluoro-phenyl)-5-(trifluoromethyl)-4H-isoxazol-3-yl]-N-[(4S)-2-ethyl-3-oxo-isoxazolidin-4-yl]-2-methyl-benzamide; the isomer (5R,4R) is 4-[(5R)-5-(3,5-dichloro-4-fluoro-phenyl)-5-(trifluoromethyl)-4H-isoxazol-3-yl]-N-[(4R)-2-ethyl-3-oxo-isoxazolidin-4-yl]-2-methyl-benzamide; and the isomer (5R,4S) is 4-[(5R)-5-(3,5-dichloro-4-fluoro-phenyl)-5-(trifluoromethyl)-4H-isoxazol-3-yl]-N-[(4S)-2-ethyl-3-oxo-isoxazolidin-4-yl]-2-methyl-benzamide. The enriched composition can comprise a molar proportion of the isomer (5S,4R) greater than 50%, e.g. at least 55%, 60%, 65%, 70%, 75%, 80%, 85%, 90%, 95%, 96%, 97%, 98% or 99%, over the total amount of the isomers (5S,4R), (5S,4S), (5R,4R) and (5R,4S).

The compound of formula XII can be prepared for example according to WO2011/067272 or to WO2016/023787, which are incorporated herein by reference.

More particularly, the process for the preparation of a compound of formula XII is performed by reacting the compound of formula XI as described in the present invention, with hydroxylamine or its salt, a base, a chiral catalyst, and an organic solvent.

The term “hydroxylamine” means the free hydroxylamine of formula H₂NOH, and the hydroxylamine salts can be for example hydroxylammonium chloride.

The chiral catalyst can be more particularly a catalyst comprising at least one chiral moiety, and preferably at least two chiral moieties. The chiral catalyst can comprise any suitable chiral catalyst well-known in the art. In a first example, the chiral catalyst can be the compounds of formula III described on page 2 in WO2016/023787 (incorporated by reference), preferably the dimeric chiral catalyst of formula III described on page 4 in WO2016/023787, and more preferably the compound R-(6-methoxy-4-quinolyl)-[(2S)-1-[[2,3,5,6-tetrafluoro-4-[(2S)-2-[(R)-hydroxy-(6-methoxy-4-quinolyl)methyl]-5-vinyl-quinuclidin-1-ium-1-yl]methyl]phenyl]methyl]-5-vinyl-quinuclidin-1-ium-2-yl]methanol dibromide (TFBBQ) with the following CAS number: 1879067-61-4 described as compound of formula XVII on page 8 in WO2016/023787. In WO2016/023787 pages 7-8, said compound of formula XVII can be prepared from the compound of formula XV with a suitable halogenating reagent such as SOBr₂, POBr₃, PBr₃, HBr, NaBr/H₂SO₄, or any combinations thereof; in a suitable solvent such as acetic acid, toluene, xylene, chlorobenzene, dichlorobenzene, heptane, ethyl acetate, dichloromethane, tetrahydrofuran, 2-methyltetrahydrofuran, 1,4-dioxane, dimethylformamide, N-methyl pyrrolidone, water, or any combinations thereof; to yield the compound of formula XVI. Then the compound of formula XVI can react with the compound of formula X described on page 7 of WO2016/023787, in the presence of a suitable organic solvent such as toluene, acetonitrile, acetone, methanol, ethanol, 1-pentanol, tetrahydrofuran, 2-methyltetrahydrofuran, 1,4-dioxane, dimethyl formamide, N-methyl pyrrolidone, anisole, water, or any combinations thereof, to yield the compound of formula XVII. In a second example, the chiral catalyst can be the compounds of formula 2 to 12 as chiral phase transfer catalysts, described in US2014350261A1 (incorporated by reference). In a third example, the chiral catalyst can be the compounds of formula III described in WO2020/094434 (incorporated by reference) or described in WO2021/197880 (incorporated by reference).

The organic solvent used in the reaction from the compound of formula XI to the compound of formula XII can be for example dichloromethane, 1,2-dichloroethane, toluene, chlorobenzene, chloroform, tert-butyl methyl ether, iso-propanol, ethanol, tetrahydrofurane, 2-methyltetrahydrofurane, acetonitrile, propionitrile, 2-methylpropionitrile, butyronitrile preferably 1,2-dichloroethane, 2-methyltetrahydrofurane, acetonitrile or dichloromethane at a temperature of between −78° C. to 60° C., preferably between −20° C. and +20° C., and at a dilution of e.g. between 0.1 M to 1 M. The reaction time is usually between 30 minutes and 48 hours, preferably between 1 and 4 hours. The amount of catalyst can be usually from 0.01 to 0.4 molar equivalents, preferably from 0.02 to 0.2 molar equivalents. The amount of hydroxylamine can be from 1 to 10 equivalents, preferably from 1.0 to 1.2 equivalents.

The base used in the reaction from the compound of formula XI to the compound of formula XII can include alkali hydroxides such as lithium hydroxide, sodium hydroxide or potassium hydroxide, preferably sodium hydroxide, in usual amounts of between 0.05 and 2 equivalents. Preferably the amount of base used is from 0.05 to 1.0 equivalents. The reaction may be carried out in the presence of water. The expression “molar equivalents” to obtain the compound of formula XII is based on the number of moles (mol) of the compound of formula XI.

The invention will now be described by way of non-limiting examples.

### EXAMPLES

### Example 1: Preparation of Potassium (4R)-2-oxooxazolidine-4-carboxylate Using Potassium Methoxide in Methanol

A double jacketed reactor equipped with a mechanical stirrer, thermometer and reflux condenser was charged with a solution of potassium methoxide in methanol (390.9 g, 1.80 mol, 32%) under nitrogen atmosphere. D-Serine (160.0 g, 1.50 mol, 98%) was added in 2 portions with an interval of 5 minutes while stirring. The resulting suspension was heated to 50° C. followed by the addition of dimethyl carbonate (150.0 g, 1.70 mol, 99%) within 1 h while keeping the temperature at 50° C. The reaction mixture was stirred at 50° C. for 2 h to result in ≥98% conversion (by ¹H NMR-analysis in DMSO-d6 spiked with a few drops of methanol). The suspension was cooled to 0° C. within 1 h then left overnight at 0° C. while stirring. Next day the reaction mixture was filtered, the filter cake was washed with cold methanol (94 g) and dried in a drying oven overnight at 50° C. resulting in (4R)-2-oxooxazolidine-4-carboxylate as a white nicely flowing non-hygroscopic crystalline powder (252.5 g). The chemical purity was 96% (by quantitative ¹H NMR in D₂O with maleic acid as standard) and the isolated yield was 96%.

¹H NMR (400 MHZ, D₂O) δ ppm: 4.63-4.69 (m, 1H), 4.48-4.55 (m, 2H), N—H is not visible due to exchange with deuterium.

¹H NMR (400 MHZ, CD₃OD) δ ppm: 4.56-4.60 (m, 1H), 4.35-4.39 (m, 1H), 4.18-4.22 (m, 1H). N—H is not visible due to exchange with deuterium.

¹³C NMR (100.6 MHz, D₂O) δ ppm: 177.9, 161.9, 69.2, 55.9.

### Example 2: Preparation of Sodium (4R)-2-oxooxazolidine-4-carboxylate Using Sodium Methoxide in Methanol

A screw cap septum vial was charged with D-Serine (1.05 g, 10.0 mmol) and methanol (2.0 mL) under nitrogen atmosphere. To the resulting suspension a solution of sodium methoxide in methanol (2.17 g, 12.0 mol, 30%) was added within 1 min at room temperature. The mixture was stirred for 10 min to result in a clear solution to which dimethyl carbonate (1.3 mL, 1.36 g, 15.0 mmol) was added in one portion. The reaction mixture was stirred at room temperature for 2 h then at 50° C. for additional 2 h. ¹H NMR analysis of the reaction mixture (400 MHZ, DMSO-d6 added with few drops of MeOH) indicated complete consumption of D-Serine (sodium salt) and 75% conversion of the intermediate carbamate (compound of formula Ia) to sodium (4R)-2-oxooxazolidine-4-carboxylate.

### Example 3: Preparation of Sodium (4R)-2-oxooxazolidine-4-carboxylate Using Sodium Ethoxide in Ethanol

A screw cap septum vial was charged with D-Serine (1.06 g, 10.0 mmol) and ethanol (2.0 mL) under nitrogen atmosphere. To the resulting suspension a solution of sodium ethoxide in ethanol (4.5 mL, 3.89 g, 12.0 mol, 21%) was added at room temperature. The mixture was stirred for 10 min followed by the addition of dimethyl carbonate (1.3 mL, 1.36 g, 15.0 mmol). The reaction mixture was stirred at room temperature overnight. ¹H NMR analysis of the reaction mixture (400 MHZ, DMSO-d6 with few drops of MeOH) indicated complete consumption of D-serine (sodium salt) and 48% conversion of the intermediate carbamates (compound of formula Ia) to sodium (4R)-2-oxooxazolidine-4-carboxylate.

### Example 4: Preparation of (4R)-2-oxooxazolidine-4-carboxylic acid from D-Serine Without Isolating potassium (4R)-2-oxooxazolidine-4-carboxylate

A double jacketed reactor equipped with a mechanical stirrer, thermometer and Dean-Stark apparatus was charged with a solution of potassium methoxide in methanol (126.0 g, 0.575 mol, 32%) under nitrogen atmosphere. D-Serine (53.7 g, 0.50 mol, 98%) was added and the resulting suspension was heated to 50° C. followed by the addition of dimethyl carbonate (47.8 g, 0.525 mol. 99%) within 0.5 h while keeping the temperature at 50° C. The reaction mixture was stirred at 50° C. for 2 h to result in ≥98% conversion (by quantitative ¹H NMR-analysis in DMSO-d6 spiked with a few drops of methanol). The reaction mixture was neutralized to pH=7-8 (wetted pH indicator paper) with concentrated sulfuric acid (3.8 g, 0.0375 mol, 98%). Methanol was replaced with methyl isobutyl ketone, first by distilling off a part of methanol (53 g, about ⅓ of the total amount) followed by continuous addition of methyl isobutyl ketone while keeping distilling off methanol (temperature in the reactor Tᵣ from 65 to 80° C.; vacuum from 1000 to 300 mbar). Totally 224 g of methyl isobutyl ketone was introduced. The residual amount of methanol in the reaction mixture constituted 1 w % (¹H NMR analysis). The mixture was cooled to 50° C. and aqueous HCl (69.1 g, 0.588 mol, 31%) was added within 10 min. The resulting mixture was dehydrated by azeotropic water removal (Temperature in the reactor Tᵣ from 55 to 63° C.; vacuum at 300 mbar). After relieving the vacuum, acetone (121 g) was added, and the mixture was heated to 64° C. The suspension of salts was filtered (hot filtration), the filter cake was washed with hot acetone (43 g) and the combined mother liquor was placed back into the reactor. Acetone was distilled off from the mixture, first at ambient pressure then under vacuum (from 1000 to 300 mbar) to cause the product to crystallize. The resulting suspension was cooled to room temperature and filtered. The cake was washed with methyl isobutyl ketone (42 g) and dried in a drying oven overnight at 50° C. to afford (4R)-2-oxooxazolidine-4-carboxylic acid as a white crystalline solid (61.6 g). The chemical purity was 93.4% (by quantitative ¹H NMR in D₂O with maleic acid as standard) and the isolated yield was 88%.

¹H NMR (400 MHZ, D₂O) δ ppm: 4.60-4.64 (m, 1H), 4.44-4.53 (m, 2H), N—H and COOH are not visible due to exchange with deuterium.

¹H NMR (400 MHZ, DMSO-d6) δ ppm: 13.21 (br s, 1H), 8.12 (s, 1H), 4.44-4.51 (m, 2H), 4.27-4.36 (m, 2H).

¹³C NMR (100.6 MHZ, D₂O) δ ppm: 174.3, 161.5, 67.8, 53.9.

### Example 5: Preparation of (4R)-2-oxooxazolidine-4-carboxylic acid from potassium (4R)-2-oxooxazolidine-4-carboxylate Using Gaseous HCl

Potassium (4R)-2-oxooxazolidine-4-carboxylate (75.1 g, 0.431 mol, 97%) was suspended in methyl isobutyl ketone (92 g) in a double jacketed reactor equipped with a mechanical stirrer, thermometer, distillation set-up and pipe for gas introduction. A stream of hydrogen chloride (25.0 g, 0.517 mol) was introduced under the liquid surface within 15 min while keeping the temperature at 20° C. The reaction mixture was stirred for 20 min at 20° C. To remove the excess of HCl, a stream of nitrogen was passed through the reaction mixture during 30 min. Acetone (115 g) and water (4 g) were added then the mixture was heated to 62° C. The suspension of salts was filtered (hot filtration), the filter cake was washed with hot acetone (28 g) and the combined filtrate was placed back into the reactor. Acetone was distilled off from the mixture, first at ambient pressure then under vacuum (from 1000 to 300 mbar) to cause the product to crystallize. The resulting suspension was cooled to room temperature and filtered. The cake was washed with methyl isobutyl ketone (51 g) and dried in a drying oven overnight at 50° C. to afford (4R)-2-oxooxazolidine-4-carboxylic acid as a white crystalline solid (49.3 g). The chemical purity was 93.7% (by quantitative ¹H NMR in D₂O with maleic acid as standard) and the isolated yield was 82%.

NMR data: ¹H NMR (400 MHZ, D₂O) δ ppm: 4.62-4.67 (m, 1H), 4.47-4.55 (m, 2H), N—H and COOH are not visible due to exchange with deuterium.

### Example 6: Preparation of (4R)-2-oxooxazolidine-4-carboxylic acid from potassium (4R)-2-oxooxazolidine-4-carboxylate Using Concentrated H₂SO₄ and 2-pentanone as Solvent

A double jacketed reactor equipped with a mechanical stirrer, thermometer and reflux condenser was charged with potassium (4R)-2-oxooxazolidine-4-carboxylate (50.0 g, 0.288 mol, 97.3%). Wet 2-pentanone (120 g, 3 w % water) prepared from 2-pentanone (116.4 g) and water (3.6 g) was added. The resulting suspension was heated to 80° C. followed by dropwise addition of concentrated sulfuric acid (28.8 g, 0.288 mol, 98%) while keeping the temperature in the rage of 80-85° C. The suspension of salts was filtered (hot filtration), the filter cake was washed with hot 2-pentanone (63 g) and the combined filtrate was placed back into the reactor. About ⅓ of the total amount of 2-pentanone (120 g) was distilled off in vacuum (from 340 to 240 mbar). The solution was cooled from 73° C. to 25° within 2 h to cause the product to crystallize. The resulting suspension was stirred overnight at 25° C. and filtered. The cake was washed with 2-pentanone (16 g) and dried in a drying oven overnight at 60° C. to afford the title compound as a slightly yellow crystalline solid (20.9 g). The chemical purity was 90.4% (by quantitative ¹H NMR in D₂O with maleic acid as standard) and the isolated yield was 50%. 37% of the theoretical amount of (4R)-2-oxooxazolidine-4-carboxylic acid was found in the salts and in the mother liquor.

¹H NMR (400 MHZ, D₂O) δ ppm: 4.62-4.67 (m, 1H), 4.47-4.56 (m, 2H), N—H and COOH are not visible due to exchange with deuterium.

1. A process for the preparation of a compound of formula II 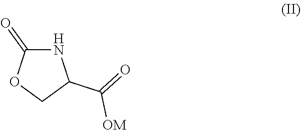
wherein M is selected among Na, K and Li,
by reacting a compound of formula I 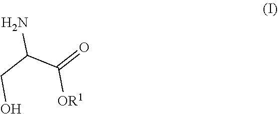
wherein R¹ is selected among hydrogen, Na, K and Li,
with a base, a reagent, and optionally an organic solvent, characterized in that the base is a metal salt of alkoxide.

2. The process according to claim 1, characterized in that the metal salt of alkoxide is an alkali metal salt of C₁-C₅ alkoxide.

3. The process according to claim 1, characterized in that the reagent is selected among an organic carbonate, a halo-carbonate, and any mixture thereof.

4. The process according to claim 1, characterized in that the reagent is selected among an aryl-carbonate, an alkyl-carbonate, an aryl-alkyl-carbonate, and any mixture thereof.

5. The process according to claim 1, characterized in that the reagent is a chloro-carbonate.

6. The process according to claim 1, characterized in that the organic solvent is selected among methanol, ethanol, propanol, isopropanol, butanol, t-butanol, t-amyl alcohol, toluene, tetrahydrofuran, and any mixture thereof.

7. The process according to claim 1, characterized in that the amount of the base is from 0.01 to 10 molar equivalents, preferably from 0.05 to 5 molar equivalents, preferably from 0.1 to 2.0 molar equivalents, and more preferably from 0.1 to 1.5 molar equivalents.

8. The process according to claim 1, characterized in that the amount of the reagent is from 0.1 to 10 molar equivalents, preferably from 0.5 to 5 molar equivalents, preferably from 0.5 to 2.0 molar equivalents, and more preferably from 0.5 to 1.5 molar equivalents

9. The process according to claim 1, characterized in that the amount of the organic solvent is from 1 to 200 molar equivalents, preferably from 1 to 100 molar equivalents, and more preferably from 1 to 20 molar equivalents.

10. The process according to claim 1, characterized in that it further comprises a crystallisation step, and optionally then a separation step.

11. A process for the preparation of a compound of formula III 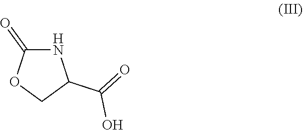
by reacting the compound of formula II with an acid in the presence of a solvent.

12. The process according to claim 11, characterized in that it further comprises a crystallisation step, and optionally then a separation step.

13. The process according to claim 11, characterized in that the acid is selected among hydrochloric acid, sulfuric acid, hydrobromic acid, trifluoroacetic acid, methane sulfonic acid, and any mixture thereof.

14. The process according to claim 11, characterized in that the solvent is selected among methyl isobutyl ketone, methyl ethyl ketone, acetone, 2-pentanone, propionic acid, acetic acid, tetrahydrofuran, 2-methyltetrahydrofuran, dioxane, methyl acetate, ethyl acetate, butyl acetate, dimethyl carbonate, ethylene carbonate and any mixture thereof.

15. A compound of formula Ia 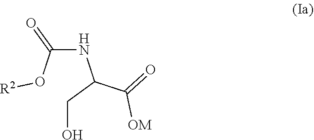
wherein R² is selected among C₁₋₄alkyl, phenyl, benzyl, C₂H₄OH, C₃H₆OH, CHCH₃CH₂OH, and CH₂CHCH₃OH; and M is selected among Na, K and Li.

16. The process for the preparation of a compound of formula VI 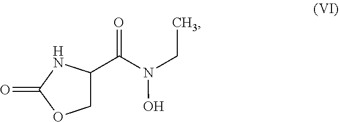 [SMILES: CCN(O)C(=O)C1COC(=O)N1]
including the process for the preparation of the compound of formula II according to claim 1.

17. The process for the preparation of a compound of formula VIII 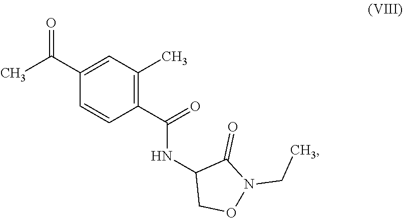 [SMILES: CCN1OCC(NC(=O)c2ccc(C(C)=O)cc2C)C1=O]
including the process for the preparation of the compound of formula II according to claim 1.

18. The process for the preparation of a compound of formula XI 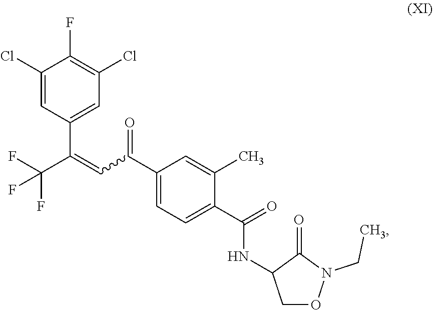 [SMILES: CCN1OCC(NC(=O)c2ccc(C(=O)C=C(c3cc(Cl)c(F)c(Cl)c3)C(F)(F)F)cc2C)C1=O]
including the process for the preparation of the compound of formula II according to claim 1.

19. The process for the preparation of a compound of formula XII or an enriched composition comprising a compound of formula XII 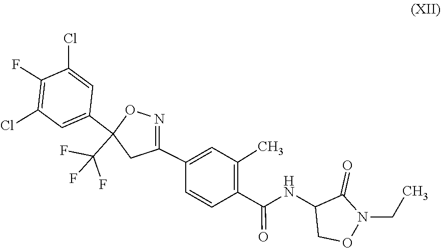 [SMILES: CCN1OCC(NC(=O)c2ccc(C3=NOC(c4cc(Cl)c(F)c(Cl)c4)(C(F)(F)F)C3)cc2C)C1=O]
including the process for the preparation of the compound of formula II according to claim 1.
